(19) United States
(12) Patent Application Publication
(10) Pub. No.: US 2024/0294726 A1
(43) Pub. Date: Sep. 5, 2024

(54) Method and Reactor System for Depolymerizing a Terephthalate-Polymer Into Reusable Raw Material

(71) Applicant: Ioniqa Technologies B.V., Eindhoven (NL)

(72) Inventors: Egor Vasilyevich FUFACHEV, Eindhoven (NL); Alexander Thomas WOLTERS, Eindhoven (NL); Andre Banier DE HAAN, Eindhoven (NL); Joost Robert WOLTERS, Eindhoven (NL)

(73) Assignee: Ioniqa Technologies B.V., Eindhoven (NL)

(21) Appl. No.: 18/572,353

(22) PCT Filed: Jun. 20, 2022
(86) PCT No.: PCT/NL2022/050347

§ 371 Date: Dec. 20, 2023

(30) Foreign Application Priority Data
Jun. 21, 2021 (NL) 2028500

(51) Int. Cl. C08J 11/24; B01D 3/14; B01D 9/00; B01J 19/00; C07C 67/475; C08J 11/16; C08J 11/26

(52) U.S. Cl. CPC C08J 11/24; B01D 3/143; B01D 9/0009; B01J 19/0033; C07C 67/475; C08J 11/16; C08J 11/26; B01D 2009/0086; B01J 2219/00186; C08J 2367/02

(57) ABSTRACT

A method and reactor system for depolymerizing a terephthalate polymer into reusable raw material are described, as well as a raw material obtainable by the method. The method inter alia comprises providing the polymer and a solvent such as ethylene glycol as a reaction mixture in a reactor. A heterogeneous catalyst, such as a metal containing particle, and/or a homogeneous catalyst is provided in the reaction mixture and the reaction mixture heated to depolymerize the polymer. Monomer comprising bis-(2-hydroxyethyl)-terephthalate (BHET), and 2-hydroxyethyl[2-(2-hydroxyethoxy)ethyl]terephthalate (BHEET) as byproduct are formed. The BHET is recovered from a depolymerized product stream exiting the reactor and a BHET-depleted stream is formed. A mass fraction of BHEET in the depolymerized product stream and/or in the BHET-depleted stream is monitored and adjusted to below a predetermined limit value of the BHEET-mass fraction in the depolymerized product stream.

### FIELD OF INVENTION

The invention relates to a method of depolymerizing a terephthalate polymer into reusable raw material, such as terephthalate monomer and oligomers. The invention further relates to a reactor system for depolymerizing a terephthalate polymer into the reusable raw material. The invention finally relates to a solid composition, being polymerizable raw material obtainable from the method of depolymerization.

### BACKGROUND

Terephthalate polymers are a group of polyesters comprising terephthalate in the backbone. The most common example of a terephthalate polymer is polyethylene terephthalate, also known as PET. Alternative examples include polybutylene terephthalate, polypropylene terephthalate, poly pentaerythrityl terephthalate and copolymers thereof, such as copolymers of ethylene terephthalate and polyglycols, for instance polyoxyethylene glycol and poly (tetramethylene glycol) copolymers. PET is one of the most common polymers and it is highly desired to recycle PET by depolymerization thereof into reusable raw material.

One preferred way of depolymerization is glycolysis, which is preferably catalyzed. Typically, as a result of the use of ethylene glycol a reaction mixture comprising at least one monomer comprising bis (2-hydroxyethyl) terephthalate (BHET) may be formed. One example of a suitable depolymerization by glycolysis is known from WO2016/105200 in the name of the present applicant. According to this process, the terephthalate polymer is depolymerized by glycolysis in the presence of a specially designed catalyst. At the end of the depolymerization process, water is added and a phase separation occurs. This enables to separate a first phase comprising the BHET monomer from a second phase comprising catalyst, oligomers and additives. The first phase may comprise impurities in dissolved form and as dispersed particles. The BHET monomer can be obtained by means of crystallization.

A high purity is required for reuse of the depolymerized raw material. As is well-known, any contaminant may have an impact on the subsequent polymerization reaction from the raw materials. Moreover, since terephthalate polymers are used for food and also medical applications, strict rules apply so as to prevent health issues.

While applicant's process according to WO2016/105200 leads to a very high conversion of the terephthalate polymer and also facilitates separation of various additives from the BHET monomer, the inventors identified by-products of the depolymerization reaction, in particular 2-hydroxyethyl[2-(2-hydroxyethoxy)ethyl]terephthalate (BHEET) and diethylene glycol (DEG) that both may have an effect on the quality of the crystallized BHET monomer.

### SUMMARY

There is a need therefore for providing a process of depolymerizing a terephthalate polymer into reusable raw material having a high purity, so as to be suitable for preparation of fresh terephthalate polymer. Such process may not always yield a very high conversion of the terephthalate polymer, but acceptable conversion (rates) may be achieved. There is also a need for providing a reactor system in which such depolymerization process may be implemented.

According to a first aspect of the invention there is provided a method of depolymerizing a polymer comprising terephthalate repeating units into reusable raw material, the method comprising the steps of:
a) providing a reaction mixture of the polymer and a solvent in a reactor, wherein the solvent is capable of reacting with the polymer and comprises or consists essentially of ethylene glycol;
b) providing a catalyst being capable of catalyzing degradation of the polymer into oligomers and/or monomers, wherein the catalyst comprises a heterogeneous catalyst, such as a metal containing particle, and/or a homogeneous catalyst;
c) forming a dispersion or solution of the catalyst in the reaction mixture;
d) heating the reaction mixture and depolymerizing the polymer in the reaction mixture using the catalyst to form a monomer comprising bis-(2-hydroxyethyl)-terephthalate (BHET), and 2-hydroxyethyl[2-(2-hydroxyethoxy)ethyl]terephthalate (BHEET) as byproduct;
e) separating the formed BHET from a depolymerized product stream exiting the reactor and comprising at least the formed BHET, BHEET and the solvent;
f) recovering a BHET-depleted stream after the separation of BHET in step e), and
g) reusing the BHET-depleted stream as at least a part of the solvent in step a) by refeeding it to the reactor,
   wherein a mass fraction of BHEET in the depolymerized product stream and/or in the BHET-depleted stream is monitored and adjusted to below a predetermined limit value of the BHEET-mass fraction in the depolymerized product stream, wherein the predetermined limit value of the BHEET-mass fraction in the depolymerized product stream defined relative to the BHET-mass fraction in the depolymerized product stream is lower than 10 wt. %, and wherein BHEET is defined by Formula I:

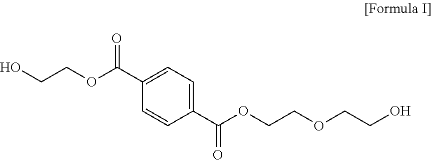 [SMILES: O=C(OCCO)c1ccc(C(=O)OCCOCCO)cc1]

According to a second aspect of the invention, a reactor system is provided for performing the method of the invention, as will be discussed in more detail below.

According to a third aspect of the invention, the invention relates to a solid composition, being polymerizable raw material obtained from depolymerization and comprising at least 90.0 wt. % BHET in crystalline form, wherein the solid composition comprises less than 5 wt. % BHEET relative to BHET.

### DETAILED DESCRIPTION OF THE INVENTION

It has been understood by the inventors in the investigations leading to the present invention that contamination of the recovered BHET, preferably recovered by crystallization, was at least in part due to the potential formation during depolymerization of 2-hydroxyethyl[2-(2-hydroxyethoxy)ethyl]terephthalate (BHEET) and also of other soluble non-volatile impurities containing ethylene glycol (EG), such as diethylene glycol (DEG), mono-2-hydroxyethyl terephthalate (MHET) and bis-2-hydroxyethyl isophthalate (iso-BHET). The presence of BHEET and/or the other impurities named in the product stream exiting the reactor and in the solution from which the BHET is recovered, preferably by crystallization, may lead to a BHET product of lesser quality in terms of crystal and other properties. It has been found that BHEET in particular is important in this respect. The present invention recognizes the importance of BHEET in particular on BHET product properties, and thus proposes to monitor and adjust a mass fraction of BHEET in the depolymerized product stream to below a predetermined limit value, such that the mass fraction of BHEET in the depolymerized product stream is below the predetermined limit value when the depolymerized product stream enters the recovery step e). As a consequence, a recovered crystalline BHET monomer product may be obtained that better meets the requirements of purity for subsequent polymerisation. It has also been established that the amount of the other soluble non-volatile impurities in the BHET monomer end product, such as DEG, MHET and iso-BHET, may also be reduced due to reduction of the amount of BHEET.

It has turned out that the catalysts used, i.e. the catalysts being capable of catalyzing degradation of the polymer into oligomers and/or monomers, wherein these catalysts comprise a heterogeneous catalyst, such as a metal containing particle, and/or a homogeneous catalyst, produce an amount of BHEET that is too high for achieving the aim of the invention at an acceptable BHET yield on large scale. The invention therefore proposes the step of adjusting the mass fraction of BHEET in the depolymerized product stream such that the mass fraction of BHEET in the depolymerized product stream is below the predetermined limit value when the depolymerized product stream enters the BHET recovery step e).

The invention thus provides a method of depolymerizing a polymer comprising terephthalate repeating units into reusable raw material, the method comprising the steps of:
a) providing a reaction mixture of the polymer and a solvent in a reactor, wherein the solvent is capable of reacting with the polymer and comprises or consists essentially of ethylene glycol;
b) providing a catalyst being capable of catalyzing degradation of the polymer into oligomers and/or monomers, wherein the catalyst comprises a heterogeneous catalyst, such as a metal containing particle, and/or a homogeneous catalyst;
c) forming a dispersion or solution of the catalyst in the reaction mixture;
d) heating the reaction mixture and depolymerizing the polymer in the reaction mixture using the catalyst to form a monomer comprising bis-(2-hydroxyethyl)-terephthalate (BHET), and 2-hydroxyethyl[2-(2-hydroxyethoxy)ethyl]terephthalate (BHEET) as byproduct;
e) separating the formed BHET from a depolymerized product stream exiting the reactor and comprising at least the formed BHET, BHEET and the solvent;
f) recovering a BHET-depleted stream after the separation of BHET in step e), and
g) reusing the BHET-depleted stream as at least a part of the solvent in step a) by refeeding it to the reactor,
   wherein a mass fraction of BHEET in the depolymerized product stream and/or in the BHET-depleted stream is monitored and adjusted to below a predetermined limit value of the BHEET-mass fraction in the depolymerized product stream, wherein the predetermined limit value of the BHEET-mass fraction in the depolymerized product stream defined relative to the BHET-mass fraction in the depolymerized product stream is lower than 10 wt. %, and wherein BHEET is defined by Formula I:

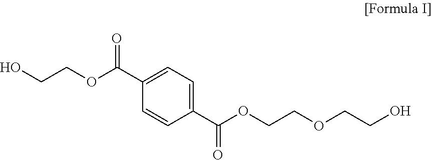 [SMILES: O=C(OCCO)c1ccc(C(=O)OCCOCCO)cc1]

The depolymerized product stream exiting the reactor comprises at least the formed BHET, BHEET, DEG and the solvent used in depolymerization. According to an embodiment of the invention, a method is provided wherein the predetermined limit value of the BHEET-mass fraction in the product stream defined relative to the BHET-mass fraction in the product stream ranges from 1 wt. % to 10 wt. %, more preferably from 2 wt. % to 9 wt. %, and most preferably from 3 wt. % to 8 wt. %.

In another embodiment of the invention, a method is provided wherein the BHEET-mass fraction in the depolymerized product stream defined relative to the BHET-mass fraction in the depolymerized product stream is lower than 10 wt. %, or, in other preferred embodiments, ranges from 0.3 wt. % to 10 wt. %, more preferably from 1 wt. % to 9 wt. %, and most preferably from 2 wt. % to 8 wt. %. Such amounts are attainable according to the invention by monitoring and adjusting the mass fraction of BHEET in the depolymerized product stream and/or in the BHET-depleted stream.

Monitoring the mass fraction of BHEET in the product stream may be achieved by any means known in the art. For instance, the mass fraction may be measured by HPLC, either in-line or performed intermittently. Samples may be taken from the product stream, for instance just after exiting the reactor, to determine the mass fraction of BHEET. The samples may also be taken from other positions in the product stream, such as just before the recovery stage of BHET. In a circular method, wherein the product stream is stripped from the BHET monomer and the remaining solvent is then refed to the reactor, it may be necessary to measure BHEET mass fraction during some cycles only. In other embodiments, the BHEET mass fraction is only monitored a number of times and then assumed for future reaction runs. Although monitoring and adjusting the amount of BHEET is performed in accordance with the invention, the invention does not exclude that monitoring and adjusting of at least one of the other impurities or by-products, such as DEG, MHET and iso-BHET, is executed as well.

It is observed, for the sake of completeness, that the adjustment of the mass fraction of BHEET in the depolymerized product stream in some embodiments may be achieved in a number of ways.

For instance, it is not excluded that the mass fraction of BHEET in the depolymerized product stream exiting the reactor is reduced by dilution with solvent and/or BHET coming from another source. The depolymerized product stream in other words may be mixed with another stream so as to arrive at conditions suitable for the recovering of BHET, preferably by crystallization and the separation of formed crystals.

In an embodiment of the method as claimed, the mass fraction of BHEET in the depolymerized product stream and/or in the BHET-depleted stream may be adjusted by removing BHEET from at least one of the named product streams to a mass fraction below the predetermined limit value in the depolymerized product stream. Removal may be performed at any stage of the method, such as from the reactor itself, between the reactor and the BHET recovery, but, preferably, downwards from the BHET recovery when a circular product stream is created in a circular process such that recovered solvent (and some BHEET) is re-fed to the reactor. The essential feature is that the mass fraction of BHEET in the depolymerized product stream is lower than a predetermined limit value before entering the BHET-recovery step e).

According to the invention a method is provided wherein the recovering step e) comprises separating BHET from the depolymerized product stream and recovering a BHET-depleted stream, and wherein the method further comprises the step of f) reusing the BHET-depleted stream as at least a part of the solvent in step a). It is not excluded that a part of the BHEET is recovered, and further processed so as to serve as a raw material for fresh polymerization for instance. Other uses may also be possible.

A further improved embodiment then adjusts the mass fraction of BHEET in the depolymerized product stream to below the predetermined limit value by purging a part of the BHET-depleted stream before refeeding it to the reactor in step g) and preferably after having recovered the BHET-depleted stream after the separation of BHET in step f).

A further embodiment offers a method wherein the purging is performed in each cycle of steps a) to g), or after each plurality of cycles of steps a) to g). The plurality of cycles may be chosen dependent on the need, and may be at least 2, more preferably at least 3, even more preferably at least 4, and at most 20, more preferably at most 15, even more preferably at most 10.

In yet another embodiment of the invention, a method is provided wherein the purging before refeeding the BHET-depleted stream to the reactor in step g) and preferably after having recovered the BHET-depleted stream after the separation of BHET in step f) is performed when a mass fraction of BHEET in the BHET-depleted stream is above a purge percentage of the predetermined limit value. The purge percentage may for instance be chosen such that it conforms to the amount of BHEET formed in one process cycle in some embodiments. This prevents the mass fraction of BHEET from accumulating in each process cycle. In such preferred embodiment, the purging is carried out until the mass fraction of BHEET in the BHET-depleted stream is about equal to the purge percentage of the predetermined limit value.

It has turned out that the purge percentage ranges from 5-50 wt % of the predetermined limit value in some embodiments. The predetermined limit value itself preferably ranges from 0-1 wt. % of the depolymerized product stream, but is more suitably defined in terms of a mass fraction relative to the mass fraction of BHET in the depolymerized product stream. The purge percentage may range from 5-20 wt % of the predetermined limit value in some embodiments.

The purging of the BHEET is preferably carried out in a distillation unit, which separates part of the BHEET from the reused solvent and optionally from water. In this process according to some embodiments, BHEET is separated from other components in the BHET-depleted stream, such as mother liquor originating from the recovery of BHET by crystallization.

The depolymerization step involves glycolysis, in which the ethylene glycol solvent is also a reactant to obtain BHET, and eventually the other by-products apart from BHEET, rather than for instance terephthalic acid that would be generated in hydrolysis. A polymer concentration in the reaction mixture or dispersion is typically from 1-30 wt. % of the total weight of the reaction mixture, although concentrations outside this range may also be possible.

The amount of ethylene glycol (EG) in the reaction mixture may be chosen within wide ranges. It has however been established that the ratio of the amount of polymer comprising terephthalate repeating units (in short PET) to the amount of EG is instrumental in influencing the BHEET mass fraction in the reaction mixture. In particular, it has been established that the BHEET mass fraction in the reaction mixture decreases with the PET:EG weight ratio. In a useful embodiment, the weight ratio of EG to the polymer is in the range of from 20:10 to 100:10, more preferably from 40:10 to 90:10, and most preferably from 60:10 to 80:10.

The reaction mixture is heated in step d) to a suitable temperature which is preferably maintained during depolymerization. The temperature may be selected in the range of from 160° C. to 250° C. It has turned out that a higher temperature in conjunction with the claimed catalyst yields a relatively low amount of BHEET in the reaction mixture and the ensuing product stream. In preferred embodiments therefore, the degrading step d) may comprise forming the monomer at a temperature in the range of from 185° C. to 225° C. Suitable pressures in the reactor are from 1-5 bar, wherein a pressure higher than 1.0 bar is preferred, and more preferably lower than 3.0 bar.

An average residence time of the BHET monomer during the degrading step d) may range from 30 sec-3 hours, and longer. In order to stop the depolymerization reaction and/or deactivate the catalyst, the temperature may be reduced to a temperature below 160° C. or lower, but preferably not lower than 85° C.

The BHET in the product stream may be recovered according to a number of methods. In a useful embodiment, the recovering step e) of BHET comprises a crystallization step wherein the depolymerized product stream is cooled, by passing through a heat exchanger for instance or, preferably, by adding water to the depolymerized product stream. In this way, a decrease of the temperature from the temperature of the degrading step d) to a crystallization temperature is achieved. Thereby BHET crystals are produced in the depolymerized product stream, thereby obtaining a mixture of BHET crystals and a mother liquor as BHET-depleted stream comprising at least ethylene glycol and BHEET. The crystallization temperature is preferably selected below 85° C., and may comprise a temperature between ambient and 85° C.

In an advantageous implementation, the crystallization temperature of the BHET crystallization is in the range of 10° C.-70° C., such as around 55° C., although lower temperatures may also be chosen, preferably in the range of 15° C.-40° C., more preferably about 18-25° C. The crystallization temperature is herein defined as the temperature defined at the start of the crystallization step, thus typically at which the nucleation occurs. It is not excluded that the temperature changes or is actively modified during the crystallization.

Yet another embodiment provides a method further comprising the step of:
recovering the mother liquor stream comprising ethylene glycol and BHEET from the product stream, and
reusing the recovered mother liquor stream as at least a part of the solvent in step a)
   wherein before the reusing step f) a part of the recovered mother liquor stream is purged when a mass fraction of BHEET in the recovered mother liquor stream is above the purge percentage of the predetermined limit value.

In another embodiment of the method, the method further comprises separating the BHET crystals from the mother liquor stream in a solid/liquid separator arranged downstream of a unit for the crystallization of BHET and upstream of a unit for purging said part of the mother liquor stream. It is also possible to use two or more units for the crystallization of BHET.

Preferably, the process conditions during the BHET crystallization are controlled. Feasible control parameters include a mass fraction of BHEET, as claimed, in the composition at the start of the step of forming the BHET crystals; and/or a volume ratio between water and ethylene glycol in the depolymerized product stream during the step of forming the BHET crystals; and/or duration of the crystallization, particularly by controlling the temperature within a predetermined range for a predefined residence time, such as 2 minutes to 120 minutes, preferably in the range of 5 minutes to 60 minutes.

Also, an anti-solvent may be added to the product stream, prior to forming BHET crystals. The anti-solvent is preferably water or an aqueous solution, such as an aqueous salt solution. The solubility of the BHET is reduced by the addition of the anti-solvent.

More generally, the process conditions may be controlled so as to control the depolymerized product stream prior to the crystallization step with respect to the mass fraction of BHEET, and also of the BHET to be crystallized, and further with respect to a volume ratio between water and ethylene glycol and the control of the temperature during a predefined period.

In accordance with other embodiments of the invention, the formation of BHET crystals precedes a solid/liquid separation step in which the corresponding mother liquor is removed and the solid BHET crystals separated therefrom. The separation step may be carried out with any method known in the art, such as by filtration.

It is not excluded that the crystallization reactor includes the separator, which is for instance activated after a predefined residence time. However, a separate separator is deemed preferable. In case that the crystals are to be recovered, a washing step is preferably carried out after the separation step. A band filter is deemed one practical arrangement for performing a separation step and a subsequent washing step. The characteristic size of the solid/liquid separation means can be chosen in dependence of the size of the generated crystals and a desired duration for the separation step. In an implementation, recovering the BHET crystals comprises separating the BHET crystals from the mother liquor by means of filtration using a filter element.

The BHET monomer is preferably recovered in solid form. It is deemed appropriate that the recovery is followed by a washing step and a drying step. Preferably, the BHET monomer crystals essentially consist of BHET, such as at least 95 wt %, more preferably at least 96 wt. % or even at least 97 wt. %. More preferably, said BHET monomer crystals comprise at most 5.0 wt % of BHEET, at most 4.0 wt % of BHEET, at most 3.0 wt % of BHEET, at most 2.0 wt % of BHEET, at most 1.5 wt % of BHEET or even at most 1.0 wt % of BHEET.

The invention may be carried out using any catalyst suitable for the purpose. Suitable catalysts include heterogeneous catalysts. In a depolymerization method according to an embodiment, the catalyst then forms a dispersion in the reaction mixture during step c). Other suitable catalysts include homogeneous catalysts. These do not form a dispersion but are typically dissolved in the reaction mixture during step c).

Several of the possible heterogeneous depolymerization catalysts are based on ferromagnetic and/or ferrimagnetic materials. Also anti-ferromagnetic materials, synthetic magnetic materials, paramagnetic materials, superparamagnetic materials, such as materials comprising at least one of Fe, Co, Ni, Gd, Dy, Mn, Nd, Sm, and preferably at least one of O, B, C, N, such as iron oxide, such as ferrite, such as magnetite, hematite, and maghemite can be used. The catalyst particles may comprise nanoparticles.

The catalyst particles catalyze the depolymerization reaction. In this depolymerization reaction individual molecules of the condensation polymer are released via a catalytic reaction out of the solid polymer, which polymer is for instance semi-crystalline. This release results in dispersing of polymer material into the reactive solvent and/or dissolving of individual polymer molecules in the reactive solvent. Such dispersing and/or dissolving is believed to further enhance depolymerization from polymer into monomers and oligomers.

One class of suitable catalysts includes the transition metals, in their metallic or ionic form. The ionic form includes free ions in solutions and in ionic bonds or covalent bonds. Ionic bonds form when one atom gives up one or more electrons to another atom. Covalent bonds form with interatomic linkage that results from the sharing of an electron pair between two atoms. The transition metal may be chosen from the first series of transition metals, also known as the 3d orbital transition metals. More particularly, the transition metal is chosen from iron, nickel and cobalt. Since cobalt however is not healthy and iron and nickel particles may be formed in pure form, iron and nickel particles are most preferred. Furthermore, use can be made of alloys of the individual transition metals.

If a catalytic particle is made of metal it may be provided with an oxide surface, which may further enhance catalysis. The oxide surface may be formed by itself, in contact with air, in contact with water, or the oxide surface may be applied deliberately.

Most preferred is the use of iron containing particles. Besides that iron containing particles are magnetic, they have been found to catalyze the depolymerization of PET for instance to conversion rates into monomer of 70-90% within an acceptable reaction time of at most 6 hours, however depending on catalyst loading and other processing factors such as the PET/solvent ratio.

Non-porous metal particles, in particular transition metal particles, may be suitably prepared by thermal decomposition of carbonyl complexes such as iron pentacarbonyl and nickel tetracarbonyl. Alternatively, iron oxides and nickel oxides may be prepared via exposure of the metals to oxygen at higher temperatures, such as 400° C. and above. A non-porous particle may be more suitable than a porous particle, since its exposure to the alcohol may be less, and therefore, the corrosion of the particle may be less as well, and the particle may be reused more often for catalysis. Furthermore, due to the limited surface area, any oxidation at the surface may result in a lower quantity of metal-ions and therewith a lower level of ions that are present in the product stream as a leached contaminant to be removed therefrom.

Another class of suitable catalysts includes particles based on earth alkali elements selected from beryllium (Be), magnesium (Mg), calcium (Ca), strontium (Sr) and barium (Ba), and their oxides. A preferred earth alkali metal oxide is magnesium oxide (MgO). Other suitable metals include but are not limited to titanium (Ti), zirconium (Zr), manganese (Mn), zinc (Zn), aluminum (Al), germanium (Ge) and antimony (Sb), as well as their oxides, and further alloys thereof. Also suitable are precious metals, such as palladium (Pd) and platinum (Pt). MgO and ZnO have been found to catalyze the depolymerization of PET for instance to conversion rates into monomer of 70-90% within an acceptable reaction time, however depending on catalyst loading and other processing factors such as the PET/solvent ratio. Suitable catalysts based on hydrotalcites are also considered.

Preferably, the catalyst particles are selected so as to be substantially insoluble in the (alcoholic) reactive solvent, also at higher temperatures of more than 100° C. Oxides that readily tend to dissolve at higher temperatures in an alcohol such as ethylene glycol, such as for instance amorphous SiO₂, are less suited.

The preferred concentration of catalyst is 1 wt % relative to the amount of PET or less. Good results have also been achieved with a catalyst loading below 0.2 wt % and even below 0.1 wt % relative to the amount of PET. Such a low loading of the catalyst is highly beneficial, and the invented method allows to recovering an increased amount of the nanoparticle catalyst.

Non-porous particles according to the invention have a surface area suitably less than 10 m²/g, more preferably at most 5 m²/g, even more preferably at most 1 m²/g. In another embodiment, the surface area is at least 3 m²/g. The porosity is suitably less than 10⁻² cm³/g or for instance at most 10⁻³ cm³/g. Porous particles may also be used, generally exhibiting a larger surface area.

Recently, quite some attention has been paid to nanoparticles as a depolymerization catalyst. Such nanoparticles have a small diameter and a surface area of in the range of from 0.1 up to 200 m²/g. Significant adsorption of the condensation polymer with these kind of nanoparticles takes place, which is believed to result in faster depolymerization and therewith an economically feasible process. To separate such nanoparticles a number of options are available.

The catalyst nanoparticle is preferably of a magnetic nature, either comprising a magnetic material, or having the ability to be magnetized sufficiently under relatively modest magnetic fields, such as being applied in the present method. Suitably, the magnetic nanoparticles contain iron, nickel and/or cobalt, in their oxidic or metallic form, or combinations thereof. Iron oxide, for instance but not exclusively in the form of Fe₃O₄ is preferred. Another suitable example is Fe:O₃. From the alloys a suitable example is CoFe:O₄. Other preferred examples are NiFe:O₄, Ni₂Fe₂O₅ or NiO. It has been found that the nanoparticles should be sufficiently small for the catalyst complex to function as a catalyst, therewith degrading the polymer into smaller units, wherein the yield of these smaller units, and specifically the monomers thereof, is high enough for commercial reasons. It has further been found that the nanoparticles should be sufficiently large in order to be able to reuse by recovering the present catalyst. It is economically unfavorable that the catalyst would be removed with either waste or degradation product obtained. Suitable nanoparticles have an average diameter in the range of from 2 up to 500 nm, more preferably in the range of from 3 up to 200 nm, even more preferably from 4 up to 100 nm. It has been found that e.g. in terms of yield and recovery of catalyst complex a rather small size of particles of 5-40 nm is optimal. It is noted that the term “size” relates to an average diameter of the particles, wherein an actual diameter of a particle may vary somewhat due to characteristics thereof. In addition aggregates may be formed e.g. in the solution. These aggregates typically have sizes in a range of 50-200 nm, such as 80-150 nm, e.g. around 100 nm. It is preferred to use nanoparticles comprising iron oxide.

Particle sizes and a distribution thereof can be measured by light scattering, for instance using a Malvern Dynamic light Scattering apparatus, such as a NS500 series. In a more laborious way, typically applied for smaller particle sizes and equally well applicable to large sizes representative electron microscopy pictures are taken and the sizes of individual particles are measured on the picture. For an average particle size, a number average may be taken. In an approximation the average may be taken as the size with the highest number of particles or as a median size.

Besides or in addition to the above-described heterogeneous catalysts, homogeneous catalysts may also catalyze the depolymerization of PET. These basic compounds most probably dissolve in the reaction mixture and act as a homogeneous system. Further examples of homogeneous catalysts include but are not limited to metal acetates, such as zinc and lithium acetate; metal carbonates, such as sodium carbonate (Na₂CO₃), metal bicarbonates, such as sodium bicarbonate (NaHCO₃); as well as metal chlorides, as is or in deep eutectic solvents. Other suitable bases that may be used comprise NaOH, CaO, KOH and KOtBu. Combinations of the above may also be used.

Other suitable catalyst may include amine containing compounds, such as trialkylamine; ionic liquids; and deep eutectic solvents. Suitable amine containing compounds are for instance disclosed in WO2015056377A1, which is expressly incorporated herein as far as the listed amine containing compounds is concerned. Deep eutectic solvents may also be used and represent a class of ionic solvents comprising two or more components, with at least two of them have a hydrogen bonding capability; one hydrogen bond donor and one hydrogen bond acceptor. The deep eutectic solvent can be a mixture of an organic salt (such as quaternary ammonium salt, e.g. choline chloride) with a metal salt (e.g. ZnCl₂, Zn(CH₃CO₂)₂, FeCl₃, etc.) or a metal salt hydrate (e.g. FeCl₂·H₂O) or a hydrogen bond donor compound (e.g. amine or carboxylic acid, such as urea); or a mixture of a metal salt with a hydrogen bond donor compound.

An ionic liquid may also be used as homogeneous catalyst. An ionic liquid generally comprises a negatively charged moiety (anion) and a positively charged moiety (cation). The cation may be aromatic or aliphatic, and/or heterocyclic. Suitable aliphatic cations may preferably be selected from guanidinium (carbamimidoylazanium), ammonium, phosphonium and sulphonium. A suitable non-aromatic or aromatic heterocyclic cation preferably comprises a heterocycle, having at least one, preferably at least two hetero-atoms. The heterocycle may have 5 or 6 atoms, preferably 5 atoms. The cation may be an aromatic moiety, which preferably stabilizes a positive charge. Typically they may carry a positive charge on the hetero-atom or the positive charge is delocalized. The hetero-atom may be nitrogen N, phosphor P or sulphur S for instance. Suitable aromatic heterocycles are pyrimidines, imidazoles, piperidines, pyrrolidine, pyridine, pyrazol, oxazol, triazol, thiazol, methimazol, benzotriazol, isoquinol and viologen-type compounds (having f.i. two coupled pyridine-ring structures). Suitable cations having N as hetero-atom comprise imidazolium, (5-membered ring with two N), piperidinium (6-membered ring with one N), pyrrolidinium (5-membered ring having one N), and pyridinium (6-membered ring with one N). Other suitable cationic moieties include but are not limited to triazolium (5-membered ring with 3 N), thiazolidium (5-membered ring with N and S), and (iso)quiloninium (two 6-membered rings (naphthalene) with N).

The anion may relate to an anionic complex, but alternatively to a simple ion, such as a halide. It may relate to a salt complex moiety, preferably a metal salt complex moiety, having a two- or three-plus charged metal ion, such as Fe³⁺, Zn²⁺, Al³⁺, Ca²⁺, and Cu²⁺, and negatively charged counter-ions, such as halogenides, e.g. Cl⁻, F⁻, and Br⁻. In an example the salt is a Fe³⁺ comprising salt complex moiety, such as an halogenide, e.g. FeCl⁴⁻. Alternatively, use can be made of counter-ions without a metal salt complex, such as halides as known per se.

It should be noted that homogeneous catalysts are more difficult to recover from the product stream. It may even be impossible to recover such catalysts. However, it could for instance be possible to recover them prior to crystallisation of the BHET monomer, but this would require special measures to overcome issues. The use of heterogeneous catalysts in the invented method is preferred therefore.

The catalyst is in preferred embodiments used in a ratio of 0.001-20 wt. %, more preferably 0.01-10 wt. %, and most preferably 0.01-5 wt. %, relative to the polymer weight.

According to another aspect of the invention there is provided a reactor system for depolymerizing a terephthalate polymer into reusable raw material, said reactor system comprising:
a depolymerization reactor comprising at least one inlet for a stream of terephthalate-containing polymer, and a stream of solvent comprising or consisting essentially of ethylene glycol and a catalyst being capable of catalyzing degradation of the polymer into oligomers and/or monomers, wherein the catalyst comprises a metal containing particle; wherein said depolymerization stage is configured for depolymerizing the terephthalate-containing polymer into a depolymerized mixture by using the ethylene glycol and the catalyst, wherein said depolymerized mixture comprises at least one monomer comprising bis (2-hydroxyethyl) terephthalate (BHET), and 2-hydroxyethyl[2-(2-hydroxyethoxy)ethyl]terephthalate (BHEET) as byproduct;
a BHET recovering stage arranged downstream from the depolymerization reactor and comprising a separator for separating BHET from a depolymerized product stream exiting the reactor and recovering a BHET-depleted stream;
a feedback loop to the reactor for reusing the BHET-depleted stream as at least a part of the solvent in the reactor, and
means for monitoring and, optionally, adjusting a mass fraction of BHEET in the depolymerized product stream and/or in the BHET-depleted stream to below a predetermined limit value of the BHEET-mass fraction in the depolymerized product stream.

This reactor system is configured for performance of the process of the invention.

The reactor system according to an embodiment is provided such that the means for adjusting the mass fraction of BHEET in the depolymerized product stream are configured to purge a part of the BHET-depleted stream before refeeding it to the reactor via the feedback loop.

Yet another embodiment provides a reactor system, comprising at least one controller unit configured to control the purging such that the mass fraction of BHEET in the BHET-depleted stream is about equal to a purge percentage of the predetermined limit value.

In another practical embodiment, a reactor system is provided wherein the BHET recovering stage comprises a crystallization unit for crystallization of BHET monomer from said product stream, wherein a remaining BHET-depleted stream constitutes a mother liquor comprising ethylene glycol and BHEET.

A preferred reactor system according to an embodiment further comprises a feedback loop to the reactor for reusing the recovered mother liquor stream as at least a part of the solvent in the reactor, and a unit for purging the mother liquor stream arranged upstream of the feedback loop when a mass fraction of BHEET in the recovered mother liquor stream is above a purge percentage of the predetermined limit value.

In such embodiment, the reactor system preferably further comprises a solid/liquid separator for separating the BHET crystals from the mother liquor stream arranged downstream of the crystallization unit for crystallization of BHET and upstream of a purging unit for purging said part of the mother liquor stream.

Another preferred embodiment relates to a reactor system, wherein the purging unit comprises a distillation unit for separating part of the BHEET from the reused solvent and optionally from water.

It may also have advantages to provide a reactor system according to yet another embodiment further comprising a separator unit for separating and recovering the catalyst complex from the product stream, and, optionally, a feedback loop to the reactor for reusing the recovered catalyst complex. A suitable separator unit may comprise one or more of a filtration unit, a centrifugation unit, or a magnetic attraction unit, or combinations of these.

Typically the BHET recovering stage comprises a crystallization unit, embodied as at least one vessel with an inlet and an outlet. Preferably a controller is present for controlling process conditions in each of said vessels. Sensors may be available thereto, as known to those skilled in the art. The crystallization unit, and the separator may be configured for batch operation or for continuous operation. Alternatively, the system is semi-continuous, in that the crystallization unit is of a batch type but the streams from the further processing stage and beyond are continuous. In this implementation, a plurality of crystallization units may be arranged in parallel so as to load one crystallization unit while performing the crystallization treatment in another parallel arranged one. In another embodiment, a plurality of crystallization units may be arranged in series for more continuous operation.

An integrated reactor system has the advantage that heat loss is reduced to a minimum, which prevents unforeseen precipitation. It is a further advantage that the mother liquor remaining after crystallization of the BHET is recycled for use in the depolymerization stage, after a certain amount of BHEET has been purged therefrom. Thereto, it is preferably subjected to a distillation treatment so as to reduce BHEET and water content in the ethylene glycol.

In an embodiment, the monomer crystal recovering stage comprises a filtration unit configured to separate the BHET crystals from the mother liquor by means of filtration, and wherein the filtration unit is configured to carry out an optional washing of the separated BHET crystals inside the filtration unit.

It is to be understood that any of the embodiments discussed hereinabove and/or hereinafter with reference to the figures or in the context of the examples or as defined in the dependent claims with respect to one aspect of the invention is also applicable and deemed disclosed in relation to any other aspect of the invention, which aspects are further defined in the claims as filed.

### BRIEF DESCRIPTION OF THE FIGURES

The above and other advantages of the features and objects of the invention will become more apparent and the invention will be better understood from the following detailed description when read in conjunction with the accompanying drawings, in which:

FIG. 1 schematically illustrates a reactor system according to an embodiment of the invention;

FIG. 2 schematically illustrates the formation of BHET monomer in time during depolymerization according to an embodiment of the invention; and

FIG. 3 schematically illustrates the formation of BHEET monomer on a logarithmic scale in time during depolymerization starting from 100 min according to an embodiment of the invention.

### DESCRIPTION OF AN EMBODIMENT

The accompanying drawings are used to illustrate presently preferred non-limiting exemplary embodiments of devices of the present invention. The figures are not drawn to scale. The same reference numerals in different figures refer to equal or corresponding elements.

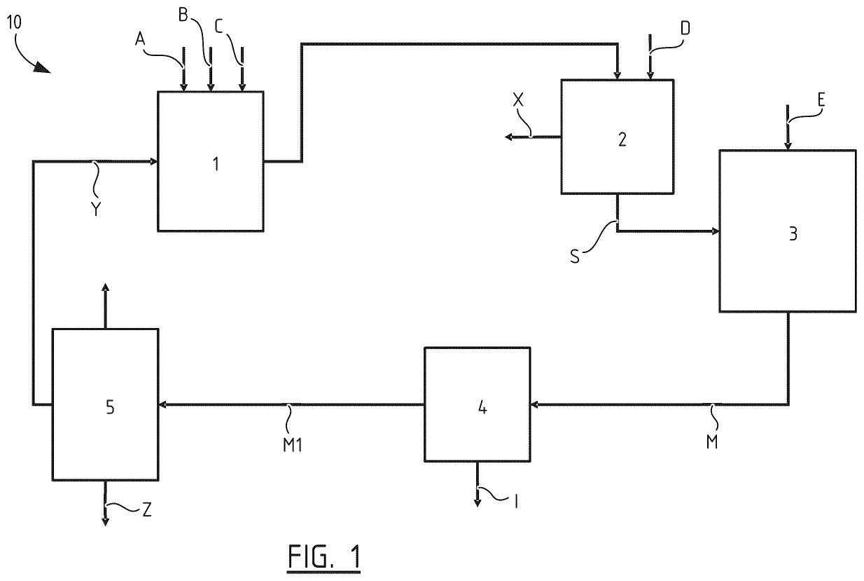

FIG. 1 illustrates schematically an embodiment of the reactor system 10 of the invention. The shown reactor system 10 essentially comprises a depolymerization reactor 1 and four separation means 2, 3, 4 and 5. Inlet streams A, B and C to the reactor 1, as well as feedback streams X and Y are indicated which respectively recycle catalyst and solvent, in particularly ethylene glycol. A purge stream Z is defined for produced BHEET. It will be understood that the FIG. 1 is a highly schematic illustration and that any variations or amendments are not excluded.

The reactor system 10 is provided with an input stream A comprising polymeric material. Preferably, this polymeric material has been pre-separated so that at least the bulk thereof is the terephthalate polymer for depolymerization, more particularly PET. The input stream A may be in solid form, such as in the form of flakes. However, it is not excluded that the input stream is in the form of a dispersion or even a solution.

The input stream A goes into the depolymerization reactor 1. Other streams entering this depolymerization reactor include a stream B of fresh solvent, such as ethylene glycol, and a stream of fresh catalyst C. The stream C may also comprise an optional recycled stream X of catalyst. A recycled stream Y of solvent, such as ethylene glycol, also enters the reactor 1. The input streams A, B, C, and the recycle streams X and Y may be arranged as individual inlets or may be combined into one or more inlets. The depolymerization reactor 1 may be of a batch type or a continuous type. While it is indicated as a single reactor, it is not excluded that a combination of reactor vessels is used, such as the combination of a tank reactor and a plurality of plug flow reactors as disclosed in WO2016/105200A1, incorporated herein by reference. Also a plurality of vessels may be arranged in parallel within one unit. While not indicated, it will be understood that the reactor system 10 is provided with a controller and that sensors may be present as well as valves for setting flow rates into the reactor and for setting residence times in the reactor. Furthermore, the reactor 1 and separation means 2, 3, 4 and 5 may be provided with heating means and/or other temperature regulation means so as to prevent deviations from predefined temperatures and other variables.

Following the depolymerization in reactor 1, the depolymerized reaction mixture is pumped to a separation/filtration unit 2, which may be provided with an inlet for water D. The water D may alternatively be provided as an aqueous solution. It is not excluded that one or more further additives are added thereto, so as to facilitate the phase separation intended to occur in the separation/filtration unit 2. The separation/filtration unit 2 serves to cool down the depolymerized mixture from a depolymerization temperature, typically in the range of 160-200° C., to a processing temperature, for instance around 100° C. The optional water D may contribute to the cooling process, and also to the generation of a two-phase mixture in the separation/filtration unit 2. A first phase at least comprises monomer BHET and BHEET as solutes in a mixture of ethylene glycol and optionally water. A second phase comprises BHET oligomers, catalyst, additives. The two-phase mixture is separated in the separation/filtration unit 2 which thereto comprises a first separator, for instance a centrifuge. The second phase containing catalyst may thereafter be recycled to the depolymerization reactor 1 as stream X. While the separation/filtration unit 2 is shown as one unit, it is not excluded that this unit 2 comprises a number of separate units, such as a cooling vessel, the first separator, and a filtration unit. Alternatively, a cooling function may actually be incorporated in the depolymerization reactor 1 as a physically single unit, particularly in case of using a batch process. Also, in other embodiments, further purification units may be provided. Separating BHEET may also be carried out before BHET crystallisation by providing a suitable separation unit for BHEET stream upwards from a BHET crystallization stage 3.

The first phase leaving the separation/filtration unit 2 is also referred to as a solution S in the context of the present invention. Rather than a pure solution, the solution S may be a colloidal solution or a dispersion. The solution S is transferred to a BHET crystallization stage 3 in which BHET is crystallized and subsequently recovered in a separator 4 as solid BHET monomer product I. Rather than or in addition to lowering the temperature relative to the separation/filtration unit 2, an anti-solvent such as water E may be added to the solution S in the crystallization stage 3, as indicated in the figure by means of the line E. This will reduce the solubility of BHET and enable crystallization and a higher temperature. Upon the crystallization of the BHET, the solution S is transformed into a slurry M that comprises solid BHET, as well as BHEET. The slurry M enters a solid/liquid separation stage 4, in which the solid BHET monomer product I is separated from the slurry M. The remaining mother liquor MI that also contains BHEET is then led to a processing stage 5, which preferably includes at least one distillation column. In the processing stage 5, the mother liquor MI is processed to reduce its water content, as well as its BHEET content through a BHEET purge Z. The resulting upgraded ethylene glycol is returned to the depolymerization reactor 1 as stream Y. The dewatering process results in a water recycle stream.

By means of the process of the invention, it has turned out feasible to arrive at a BHET monomer product I that is white and free of major contaminants.

Further variations may be envisaged by a skilled person. It is for instance feasible that the recycling of one or more of the streams X and Y comprises a (further) purification step, heating or cooling step. It is not excluded that the streams X and Y are merged prior to the entry into the depolymerization stage.

### Experiments

Depolymerization experiments were carried out using a 500 ml round bottom flask. An amount of 0.025 g of dry heterogeneous catalyst was used in combination with 50 g of polyethylene terephthalate (PET) flakes (pieces of 0.3×0.3 cm²) and 200 g of ethylene glycol (EG). An amount of 0.02 g of homogeneous zinc acetate catalyst (Zn(CH₃CO₂)₂) was used in the depolymerization reaction. The tested heterogeneous catalysts of Examples 1-5 were chosen as indicated in Table 1. A homogeneous catalyst was used in Example 3, as also shown in Table 1.

The round bottom flask was placed in the heating set up. The heating was started under stirring, and after 20 minutes, the reaction mixture had reached the reaction temperature of 197° C. under reflux. The reaction was followed in time by taking in-process-control samples to measure the mass fraction of monomer (bis(2-hydroxyethyl) terephthalate, or BHET) and by-products (such as BHEET) produced as a function of time. The mass fraction of BHET and BHEET was determined with HPLC.

TABLE 1
catalysts used

| | Catalyst | Amount (g) |
|---|---|---|
| Example 1 | Iron Oxide (Fe3O4) | 0.025 |
| Example 2 | Zinc Oxide (ZnO) | 0.025 |
| Example 3 | Zinc Acetate catalyst ((Zn(CH₃CO₂)₂ | 0.02 |
| Example 4 | Magnesium Oxide (MgO) | 0.025 |
| Example 5 | Antimony Oxide (Sb₂O₃) | about 0.025 |

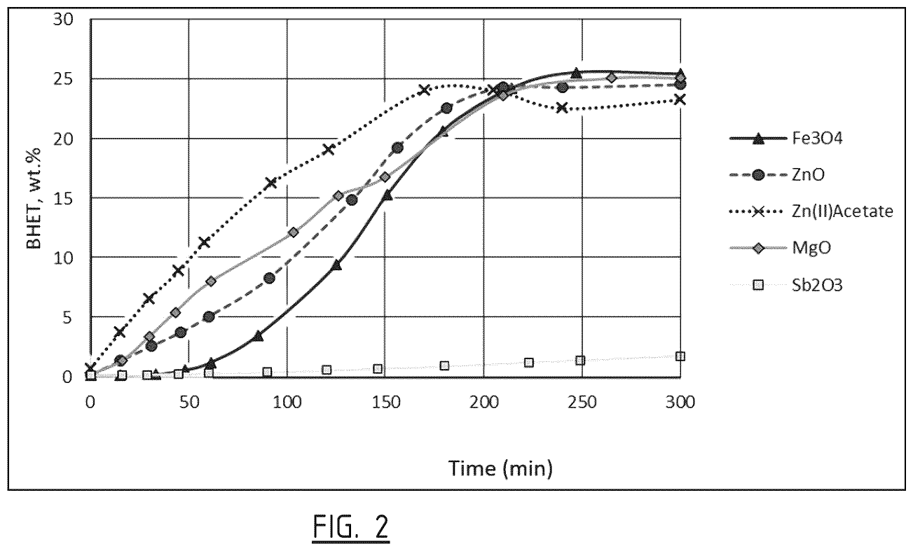

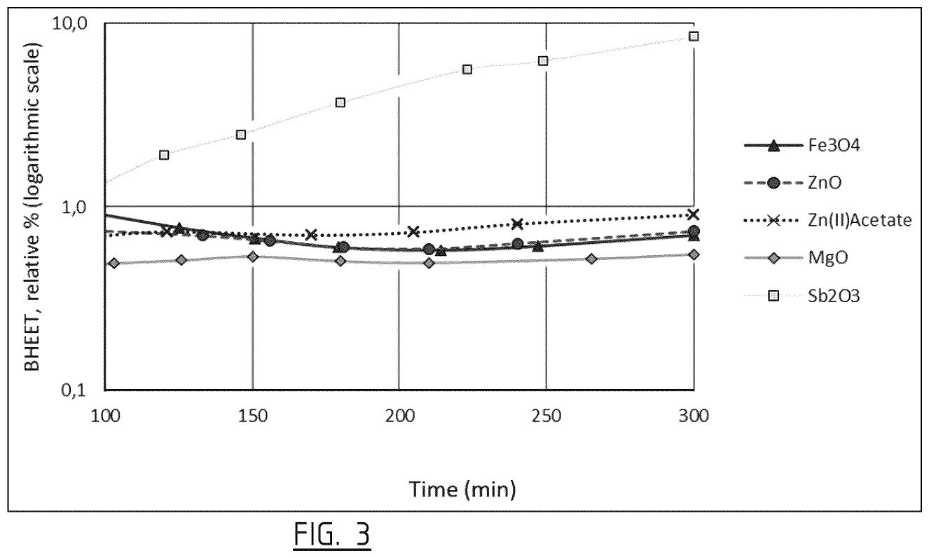

The results are shown in FIGS. 2 and 3.

FIG. 2 shows that the catalysts used in Examples 1-5 combine a relatively high depolymerization rate with an acceptable BHET formation, apart from the antimony oxide catalyst. The antimony oxide catalyst indeed performs rather badly.

FIG. 3 shows that the catalysts used in Examples 1-5 produce a relatively high amount of BHEET during depolymerization. Please note that the relative amount of BHEET produced between 100 and 300 minutes is shown on a logarithmic scale. The results mean that a BHEET purge, as claimed, is necessary for these types of catalyst. In particular the antimony oxide catalyst produces a very high amount of BHEET. For this catalyst therefore, a relatively high amount of BHEET purge is necessary.

The invention as claimed by the appended claims offers a solution for preventing impurities such as BHEET—and others like DEG, MHET and iso-BHET—from entering the BHET monomer product, resulting from the depolymerization of PET.

1. A method of depolymerizing a terephthalate polymer into reusable raw material, the polymer being a homo- or copolymer comprising a terephthalate repeating unit, the method comprising the steps of
a) providing a reaction mixture of the polymer and a solvent in a reactor, wherein the solvent is capable of reacting with the polymer and comprises or consists essentially of ethylene glycol;
b) providing a catalyst being capable of catalyzing degradation of the polymer into oligomers and/or monomers, wherein the catalyst comprises a heterogeneous catalyst, such as a metal containing particle, and/or a homogeneous catalyst;
c) forming a dispersion or solution of the catalyst in the reaction mixture;
d) heating the reaction mixture and depolymerizing the polymer in the reaction mixture using the catalyst to form a monomer comprising bis-(2-hydroxyethyl)-terephthalate (BHET), and 2-hydroxyethyl[2-(2-hydroxyethoxy)ethyl]terephthalate (BHEET) as byproduct;
e) separating the formed BHET from a depolymerized product stream exiting the reactor and comprising at least the formed BHET, BHEET and the solvent;
f) recovering a BHET-depleted stream after the separation of BHET in step e), and
g) reusing the BHET-depleted stream as at least a part of the solvent in step a) by refeeding it to the reactor,
wherein a mass fraction of BHEET in the depolymerized product stream and/or in the BHET-depleted stream is monitored and adjusted to below a predetermined limit value of the BHEET-mass fraction in the depolymerized product stream, wherein the predetermined limit value of the BHEET-mass fraction in the depolymerized product stream defined relative to the BHET-mass fraction in the depolymerized product stream is lower than 10 wt. %, and wherein BHEET is defined by Formula I:

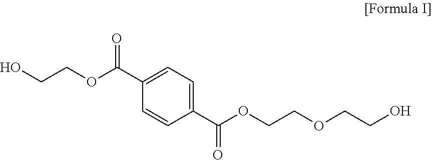 [SMILES: O=C(OCCO)c1ccc(C(=O)OCCOCCO)cc1]

2. Method as claimed in claim 1, wherein the mass fraction of BHEET in the depolymerized product stream is adjusted to below the predetermined limit value by purging a part of the BHET-depleted stream before refeeding it to the reactor in step g).

3. Method as claimed in claim 2, wherein the purging is performed in each cycle of steps a) to g), or after each plurality of cycles of steps a) to g).

4. Method as claimed in claim 2, wherein the purging is performed when a mass fraction of BHEET in the BHET-depleted stream is above a purge percentage of the predetermined limit value.

5. Method as claimed in claim 4, wherein the purging is performed until the mass fraction of BHEET in the BHET-depleted stream is about equal to the purge percentage of the predetermined limit value.

6. Method as claimed in claim 4, wherein the predetermined purge percentage ranges from 5-50 wt % of the predetermined limit value.

7. Method as claimed in claim 1, wherein the predetermined limit value of the BHEET-mass fraction in the depolymerized product stream defined relative to the BHET-mass fraction in the depolymerized product stream ranges from 0.1 wt. % to 10 wt. %.

8. Method as claimed in claim 1, wherein the recovering step e) of BHET comprises a crystallization step wherein the depolymerized product stream is cooled, preferably by adding water to the depolymerized product stream, to decrease the temperature from the temperature of the degrading step d) to below 160° C. thereby forming BHET crystals from the depolymerized product stream, thereby obtaining a mixture of BHET crystals and a mother liquor as BHET-depleted stream comprising ethylene glycol and BHEET.

9. Method as claimed in claim 8, wherein the method further comprises the step of:
recovering the mother liquor stream comprising ethylene glycol and BHEET from the depolymerized product stream, and
reusing the recovered mother liquor stream as at least a part of the solvent in step a)
wherein before the reusing step f) a part of the recovered mother liquor stream is purged when a mass fraction of BHEET in the recovered mother liquor stream is above a predetermined purge percentage of the predetermined limit value.

10. Method as claimed in claim 8, further comprising separating the BHET crystals from the mother liquor stream in a solid/liquid separator arranged downstream of a unit for the crystallization of BHET and upstream of a unit for purging said part of the mother liquor stream.

11. Method as claimed in claim 4, wherein the purging is performed in a distillation unit, which separates part of the BHEET from the reused solvent and optionally from water.

12. Method as claimed in claim 1, wherein a weight ratio of EG to the polymer in the reaction mixture is in the range of from 20:10 to 100:10, more preferably from 40:10 to 90:10, and most preferably from 60:10 to 80:10.

13. Method as claimed in claim 1, wherein a polymer concentration in the dispersion is 1-30 wt. % of the total weight of the reaction mixture.

14. Method as claimed in claim 1, wherein an average residence time of the BHET monomer during the degrading step d. is from 30 sec.-3 hours, or up to 24 hours.

15. Method as claimed in claim 1, wherein the degrading step d. comprises forming the monomer at a temperature higher than 190° C., and preferably at most 250° C., at a pressure higher than 1.0 bar, and preferably lower than 3.0 bar.

16. Method as claimed in claim 1, wherein the method further comprises the step of recovering the catalyst, preferably by separation through centrifugation and/or filtration and/or magnetic attraction.

17. Method as claimed in claim 1, wherein the catalyst comprises a metal-containing particle.

18. Method as claimed in claim 17, wherein the metal-containing particle comprises a metal oxide.

19. Method as claimed in claim 17, wherein the metal is a transition metal, preferably wherein the metal oxide is iron oxide.

20. Method as claimed in claim 19, wherein the iron oxide is magnetite (Fe₃O₄).

21. Method as claimed in claim 18, wherein the metal is an earth alkali element selected from beryllium, magnesium, calcium, strontium and barium, preferably wherein the metal oxide is magnesium oxide (MgO).

22. A reactor system for depolymerising a terephthalate polymer into reusable raw material, said reactor system comprising:
a depolymerization reactor comprising at least one inlet for a stream of terephthalate-containing polymer, and a stream of solvent comprising or consisting essentially of ethylene glycol and a catalyst being capable of catalyzing degradation of the polymer into oligomers and/or monomers, wherein said depolymerization reactor is configured for depolymerizing the terephthalate-containing polymer into a depolymerized mixture by using the ethylene glycol and the catalyst, wherein said depolymerized mixture comprises at least one monomer comprising bis (2-hydroxyethyl) terephthalate (BHET), and 2-hydroxyethyl[2-(2-hydroxyethoxy)ethyl]terephthalate (BHEET) as byproduct;
a BHET recovering stage arranged downstream from the depolymerization reactor and comprising a separator for separating BHET from a depolymerized product stream exiting the reactor and recovering a BHET-depleted stream;
a feedback loop to the reactor for reusing the BHET-depleted stream as at least a part of the solvent in the reactor, and
means for monitoring and adjusting a mass fraction of BHEET in the depolymerized product stream and/or in the BHET-depleted stream to below a predetermined limit value of the BHEET-mass fraction in the depolymerized product stream.

23. Reactor system as claimed in claim 22, wherein the means for adjusting the mass fraction of BHEET in the depolymerized product stream are configured to purge a part of the BHET-depleted stream before refeeding it to the reactor via the feedback loop.

24. Reactor system as claimed in claim 23, wherein the reactor system comprises at least one controller unit configured to control the purging such that the mass fraction of BHEET in the BHET-depleted stream is about equal to a purge percentage of the predetermined limit value.

25. Reactor system as claimed in claim 22, wherein the BHET recovering stage comprises a crystallization unit for crystallization of BHET monomer from said product stream, wherein a remaining BHET-depleted stream constitutes a mother liquor comprising ethylene glycol and BHEET.

26. Reactor system as claimed in claim 25, further comprising a feedback loop to the reactor for reusing the recovered mother liquor stream as at least a part of the solvent in the reactor, and a unit for purging the mother liquor stream arranged upstream of the feedback loop when a mass fraction of BHEET in the recovered mother liquor stream is above a predetermined purge percentage of the predetermined limit value.

27. Reactor system as claimed in claim 25, further comprising a solid/liquid separator for separating the BHET crystals from the mother liquor stream arranged downstream of the crystallization unit for crystallization of BHET and upstream of a purging unit for purging said part of the mother liquor stream.

28. Reactor system as claimed in claim 22, wherein the purging unit comprises a distillation unit for separating part of the BHEET from the reused solvent and optionally from water.

29. Reactor system as claimed in claim 22, further comprising a separator unit for separating and recovering the catalyst complex from the depolymerized product stream and, optionally, a feedback loop to the reactor for reusing the recovered catalyst complex.

30. A solid BHET composition obtainable by the method according to claim 1, comprising at least 90.0 wt. % BHET in crystalline form, wherein the solid composition comprises less than 5 wt. % BHEET relative to BHET, more preferably less than 2 wt. % BHEET relative to BHET.
